(19) United States
(12) Patent Application Publication
(10) Pub. No.: US 2007/0100355 A1
(43) Pub. Date: May 3, 2007

(54) Method and device for controlling bulking agent depth in tissue

(75) Inventors: Eric H. Bonde, Victoria, MN (US); Carole A. Tronnes, Stillwater, MN (US); Yelena G. Tropsha, Plymouth, MN (US); Lisa Ann Draper, Coon Rapids, MN (US)

Correspondence Address: MEDTRONIC, INC., 710 MEDTRONIC PARK, MINNEAPOLIS, MN, 55432-9924, US

(73) Assignee: Medtronic, Inc., Minneapolis, MN (US)

(21) Appl. No.: 11/260,828

(22) Filed: Oct. 27, 2005

(51) Int. Cl. A61B 17/34; A61B 17/94

(52) U.S. Cl. 606/108; 606/185; 623/23.68

(57) ABSTRACT

The invention includes a delivery device for introducing a prosthesis beneath a tissue surface that includes an elongate body having a proximal end and a distal end, an opening on the elongate body, which is at least partially covered by a mesh, and a deployment lumen extending through the body that has a longitudinal axis and a distal end, wherein the distal end of the deployment lumen is positioned proximal of the opening. Also included are delivery systems and kits that include delivery devices of the invention. Further, an overtube for receiving an endoscope, that includes an elongate, flexible tubular body, having a proximal end and a distal end and a longitudinal axis, at least one lumen extending therethrough for receiving an endoscope, an opening on the body, in communication with the lumen, and a tissue limiting surface within the body aligned with the opening, formed at least in part by a mesh. Methods of using the devices, and kits are also included in the invention.

### FIELD OF THE INVENTION

The present invention relates generally to the field of esophageal prosthetics. More specifically, a prosthesis delivery device and method is disclosed for submucosal insertion of a prosthetic bulking device.

### BACKGROUND OF THE INVENTION

Gastroesophageal reflux is a physical condition in which stomach acids reflux, or flow back from the stomach into the esophagus. Frequent reflux episodes (two or more times per week), may result in a more severe problem known as gastroesophageal reflux disease (GERD). Gastroesophageal reflux disease is the most common form of dyspepsia, being present in approximately 40% of adults in the United States on an intermittent basis and approximately 10% on a daily basis.

Dyspepsia, or heartburn, is defined as a burning sensation or discomfort behind the breastbone or sternum and is the most common symptom of GERD. Other symptoms of GERD include dysphasia, odynophagia, hemorrhage, water brash, and pulmonary manifestations such as asthma, coughing or intermittent wheezing due to acid aspiration.

Dyspepsia may also mimic the symptoms of a myocardial infarction or severe angina pectoris. Many factors are believed to contribute to the onset of GERD including transient lower esophageal sphincter relaxations, decreased LES resting tone, delayed stomach emptying, and an ineffective esophageal clearance. Many in the field agree, however, that the primary cause of GERD is the lack of competency of the lower esophageal sphincter.

The lower esophageal sphincter, or valve, is comprised of smooth muscle located at the gastroesophageal (GE) junction and functions to allow food and liquid to pass into the stomach but prevent regurgitation of stomach contents. At rest, the LES maintains a high-pressure zone between 10 and 30 mm Hg above intragastric pressure. Upon deglutition, the LES relaxes before the esophagus contracts, allowing food to pass through into the stomach. After food passes into the stomach, the LES contracts to prevent the stomach contents and acids from regurgitating into the esophagus. The mechanism of LES opening and closing is influenced by innervation via the vagus nerve and hormonal control of gastrin and possibly other gastrointestinal hormones.

The severity of GERD varies from patient to patient and in extreme cases complications including esophageal erosion, esophageal ulcers, and esophageal stricture are observed. Esophageal stricture is a serious condition which results from prolonged exposure of the esophageal mucosa to acid reflux. The most common clinical manifestation of stricture is dysphasia. Unlike dysphasia from non-strictured esophageal reflux, dysphasia caused by stricture is progressive in that the size of a bolus which can pass into the stomach progressively becomes smaller. In addition to esophageal erosion and ulceration, prolonged exposure of the esophageal mucosa to stomach acid can lead to a condition known as Barrett's Esophagus. Barrett's Esophagus is an esophageal disorder that is characterized by the replacement of normal squamous epithelium with abnormal columnar epithelium. This change in tissue structure is clinically important not only as an indication of severe reflux, but the appearance of columnar epithelium in the esophagus is indicative of cancer.

A relatively new option for the treatment of GERD includes the use of an esophageal bulking device that is implanted below the mucosa in the vicinity of the lower esophageal sphincter. Generally, the bulking device is made of an expandable hydrogel. Exemplary embodiments of this treatment device and method can be found in U.S. Pat. No. 6,098,629; 6,338,345; 6,401,718; and 6,725,866, the disclosure of which are incorporated herein by reference. It has also been suggested that these types of devices and methods could be used for the treatment of other conditions that may or may not be associated with a sphincter. Examples of such treatments include the treatment of urinary incontinence (US Patent Publication No. 2005/0096751); and the treatment of fecal incontinence (US Patent Publication No. 2005/0096497). In the use of such devices and/or methods, it can often be difficult to precisely control the depth of implantation of the bulking device. For this reason, there remains a need for a delivery device and method of implanting such bulking devices that afford more precise control of the depth of implantation of the bulking device.

### SUMMARY OF THE INVENTION

An embodiment of the invention also includes a delivery device for introducing a prosthesis beneath a tissue surface that includes an elongate body having a proximal end and a distal end, an opening on the elongate body, the opening being at least partially covered by a mesh, and a deployment lumen extending through the body, the deployment lumen having a longitudinal axis and a distal end, wherein the distal end of the deployment lumen is positioned proximal of the opening.

An embodiment of the invention also includes a delivery system for delivering an expandable sub-mucosal bulking agent that includes a delivery device of the invention, a tubular cannula having a sharpened distal end, positioned within the elongate body, the cannula axially moveable such that the sharpened distal end can penetrate tissue stabilized by the opening of the delivery device, and a plunger movably positioned within the cannula for ejecting a bulking agent positioned within the cannula, wherein the plunger is distally advanceable beyond the distal end of the sharpened tip.

An embodiment of the invention also includes a kit that includes a delivery system of the invention and at least one bulking agent.

An embodiment of the invention also includes an overtube for receiving an endoscope, that includes an elongate, flexible tubular body, having a proximal end and a distal end and a longitudinal axis, at least one lumen extending therethrough for receiving an endoscope, an opening on the body, in communication with the lumen, and a tissue limiting surface within the body aligned with the opening, formed at least in part by a mesh.

An embodiment of the invention includes a method of stabilizing a tissue site and introducing a prosthesis beneath the tissue, that includes the steps of providing an elongate, flexible, tubular device, having a side opening near a distal end thereof, positioning the side opening at the site, applying suction to the device to draw tissue laterally into the side opening, limiting lateral advance of the tissue into the device at least in part by contacting the tissue with mesh, and introducing a prosthesis beneath the surface at the site while the tissue is in contact with the mesh.

### BRIEF DESCRIPTION OF THE DRAWINGS

FIG. 1 is a schematic view of a pair of gastroesophageal bulking prostheses in accordance with the present invention, positioned at about the junction between the esophagus and the stomach, before expansion, in which the LES is in an open configuration.

FIG. 2 is a schematic view as in FIG. 1, following expansion of the prostheses, in which the LES is illustrated in a closed configuration.

FIG. 3A is a side elevational schematic view of an overtube in accordance with the present invention, for receiving an endoscope to deliver a submucosal prosthesis.

FIG. 3B is a bottom view of the overtube illustrated in FIG. 3A.

FIG. 4 is a side elevational schematic view of a bulking device deployment catheter, such as for “blind” deployment.

FIG. 5A is a side elevational cross section through a portion of an exemplary device depicted by the axes A and B in FIG. 3B.

FIG. 5B is a perspective schematic view of the same portion of the device depicted in FIG. 5A.

FIG. 6 is a side elevational cross section through a portion of an exemplary device depicted by the axes A and B in FIG. 3B.

FIG. 7A is a side elevational cross section through a portion of an exemplary device depicted by the axes A and B in FIG. 3B.

FIG. 7B is an example of a perspective schematic view of the same portion of the device depicted in FIG. 7A.

FIG. 7C is another example of a perspective schematic view of the same portion of the device depicted in FIG. 7A.

FIG. 7D is another example of a perspective schematic view of the same portion of the device depicted in FIG. 7A.

FIG. 8 is a side elevational schematic view of a prosthesis delivery needle assembly.

FIG. 9 is an enlarged cross sectional view of the distal end of the delivery needle of FIG. 8.

FIG. 10 is a side elevational view of the distal end shown in FIG. 9 with the plunger distended to eject a bulking agent.

FIG. 11 is a side elevational view of an alternate distal end of a deployment catheter, adapted to deploy an inflatable balloon.

### DETAILED DESCRIPTION OF THE INVENTION

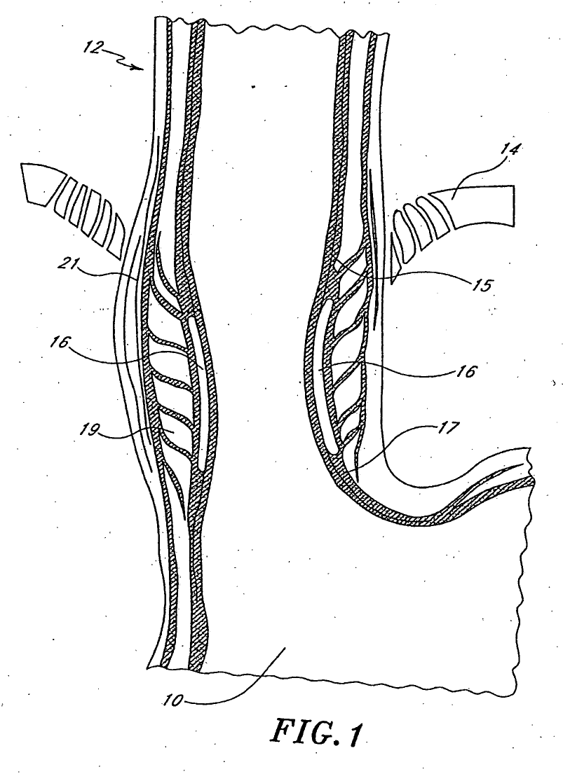

Referring to FIG. 1, there is illustrated a schematic representation of the stomach 10 and a portion of the lower esophagus 12. The esophagus 12 extends through the diaphragm 14, below which the esophagus 12 communicates with the interior of the stomach 10. A pair of gastroesophageal prosthetic bulking devices 16, in accordance with the present invention, is illustrated at about the junction between the lower esophagus 12 and the stomach 10.

In the illustrated embodiment, the bulking device 16 is delivered in the submucosa 17. The submucosa 17 is a fibrous layer of tissue positioned between the mucosa 15 and a layer of circular muscle 19. The circular muscle 19 is surrounded by a layer of longitudinal muscle 21, as is well understood in the art. In one embodiment, the bulking device 16 is delivered beneath the mucosa 15 as is discussed elsewhere herein. The bulking device 16 may either be delivered within the submucosa 17 as illustrated, or at the interface of adjacent tissue planes, such as between the mucosa 15 and submucosa 17, or between the submucosa 17 and circular muscle 19. In one embodiment, the bulking device 16 is positioned radially inwardly from the circular muscle layer 19.

Although the anatomy illustrated in FIG. 1 is generally normal, except for the improperly functioning native lower esophageal sphincter, the present invention is also useful in patients having lower esophageal abnormalities, such as hiatal hernia. In this condition, a portion of the wall of the stomach 10 extends upwardly through the diaphragm 14 and herniates superiorly to the diaphragm 14. The existence of a hiatal hernia or another abnormality in the lower esophagus may affect the delivered position of the esophageal prosthetic bulking device 16, but may not disqualify a patient otherwise treatable with the prosthetic bulking device 16 of the present invention.

As illustrated in FIG. 1, the esophageal bulking device 16 is generally delivered below the mucosa such as in the submucosa or between adjacent tissue planes in the vicinity of the sphincter. The submucosa is a springy tissue, which needs to be expanded in order to produce a cavity for delivery of the bulking device 16.

Ideally, the esophageal bulking device 16 is delivered in a position that extends across or is closely adjacent to the sphincter so that residual sphincter activity is optimized and the mucosal regions of the esophagus are protected from acid reflux. The precise positioning of the prosthesis 16 depends largely on the patient's anatomy and the severity of GERD, and will be a matter of clinical choice at the time of delivery. In patients with a hiatal hernia, for example, the esophageal bulking device 16 is delivered as close as possible to the sphincter but care must be taken to insure that the hernia will not perturb the operation of the bulking device 16.

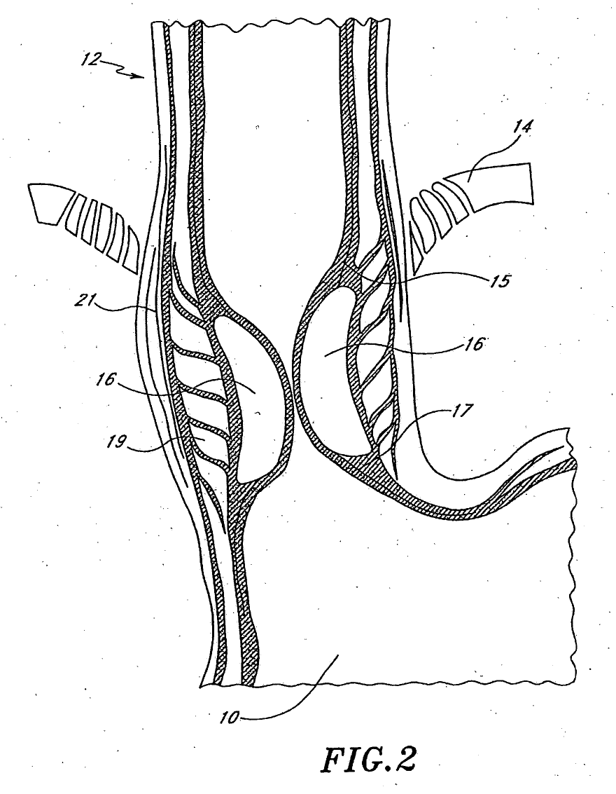

Advantageously, the esophageal bulking device 16 of the present invention enhances any residual closing pressure function of the sphincter so as to effectively reduce or prevent the reflux of stomach contents into the esophagus. In an open or relaxed state, as shown in FIG. 1, the esophageal bulking device allows food and liquid to pass through the esophagus into the stomach. However, when the sphincter is closed, as shown in FIG. 2, the esophageal bulking device increases the closing pressure along a sufficient axial length that, in cooperation with the contraction of the sphincter, it inhibits or prevents the reflux of stomach contents.

Depending on the degree of LES dysfunction, more than one esophageal bulking device 16 can be delivered into the lining of the esophagus. Where two or three or more esophageal bulking devices 16 are utilized to improve LES function, the bulking devices 16 may be spaced around the circumference of the LES. Generally, multiple bulking devices 16 will be located substantially in the same transverse plane perpendicular to the longitudinal axis of the esophagus. Use of multiple bulking devices 16 without rigid interconnection permits the LES to radially expand to a greater degree so as to permit bolus passage and better accommodate the natural function of the esophagus.

Depending on the patient's anatomy and the extent of GERD, esophageal bulking devices 16 having various lengths and cross sectional profiles are delivered so as to maximize operation with the residual LES function. Using common esophageal manometry and endoscopic techniques, for example, medical personnel can measure the degree of closure (e.g., closing pressure) achieved by the sphincter as well as any unique anatomical features of the lower esophagus for a particular patient when selecting specific shapes and designs of esophageal bulking devices 16 to be delivered.

Due to the irregular cross-sectional configuration of the closed sphincter, the cross-sectional configuration of the bulking device 16 can take on any of a wide variety of shapes including, but not limited to, those specific embodiments disclosed herein. In general, the desired expanded transverse plane thickness of the esophageal bulking device 16 will depend on the lumen diameter and the extent of LES dysfunction. Devices 16 produced in accordance with the present invention can be rated according to their bulking area, which represents the total cross-sectional area the device will occupy within the region at or about to the sphincter, referred to as the “bulking zone”. In general, a larger transverse cross-sectional area will produce a higher closing pressure for a given state of the disease.

Exemplary configurations of an esophageal bulking device 16 generally include oblong, cylindrical, elliptical, toric or pillow shapes. In one embodiment, a proximal portion of the bulking device can have a smaller cross-sectional area than a distal portion. Alternatively, embodiments of the esophageal bulking device 16 can be a cylindrical shape or elliptical wherein a proximal portion and a distal portion have roughly the same cross-sectional area and shape. The bulking device 16 preferably substantially retains its pre-implanted configuration once delivered into the body.

Suitable esophageal bulking devices 16 comprise a soft, flexible body that may have an expanded axial length from 0.5 cm to 5.0 cm, a width (circumferential direction) of 0.2 cm to 2.0 cm, and a thickness (radial direction) of 0.2 cm to 2.0 cm. Many esophageal bulking devices 16 of the present invention have a length within the range of 1.0 cm to 4.0 cm, a width within the range of 0.2 cm to 1.5 cm, and a thickness within the range of 0.2 cm to 1.5 cm. In one embodiment, the esophageal bulking device 16 has a length of 2.0 cm to 3.0 cm, a width of 0.8 cm to 1.0 cm, and a thickness of 0.4 cm to 0.6 cm. The cross-sectional configuration may be circular, oval or other configuration, as desired.

Length to thickness ratios are generally no more than about 15:1 and are often no more than about 6:1 or 4:1. Length to thickness ratios on the order of less than 3:1 may also be desirable depending upon the severity of the condition. The cross-sectional area of the bulking device 16 may also vary at different points along the length of the same device 16. As mentioned above, optimal dimensions may be patient specific and can be determined through routine experimentation of one of skill in the art in view of the disclosure herein.

An LES having a relaxed open diameter of 2.0 cm, for example, has a cross-sectional lumen area of 3.14 cm.sup.2. A 25% bulking function could be accomplished by providing a bulking device 16 having a total cross-sectional area in the bulking zone of about 0.785 cm.sup.2. The bulking area may represent the area of an esophageal bulking device 16 having a generally oval or rectangular cross-section (e.g., 0.443 cm.times. 1.772 cm) which is adapted to extend axially for a length of 1 to 3 cm beneath the mucosa.

In general, the objective of the present invention is to increase the closing pressure of the lower esophageal sphincter. The present inventors believe that a closing pressure of at least a certain minimum closing threshold value, maintained along a minimum axial effective LES length will satisfactorily reduce esophageal reflux. In the intra-abdominal (i.e., inferior to the diaphragm 14) esophagus, about 2 cm of effective LES length appears desirable. An average pressure along that length is preferably in excess of about 10 mm Hg, preferably at least about 15 mm Hg, and optimally in the range of from about 15 mm to about 30 mm Hg.

Within certain outer limits, any increase in the closing pressure in the LES may improve symptoms. For example, some patients have an LES closing pressure on the order of about 5 mm Hg, which is accompanied by severe GERD symptoms. At the high end, a closing pressure in excess of about the minimum diastolic pressure inhibits blood flow, thereby increasing the risk of localized pressure necrosis. Pressure slightly below the minimal diastolic pressure may still interfere with swallowing function. The present invention therefore preferably enables increasing the closure pressure from a pretreatment value below about 10 to 15 mm Hg to a post treatment value of preferably on the order of from about 18 or 20 to about 25 or 30, along a length of at least about 1.0 cm and preferably at least about 2 cm or 2.5 cm or more.

Once the total desired cross-sectional area and length of the bulking device is determined for a particular patient or class of patients, the allocation of that cross-sectional area to a single bulking device or a series of bulking devices which together produce the desired cross-sectional area must be determined. This clinical decision will take into account any unique aspects to the patients lower esophageal anatomy, together with the extent of the disease and consequent total area of bulking required. In general, a larger single bulking device will require a larger submucosal pocket for delivery and the consequent greater disruption of tissue in the immediate area of the prosthesis, which may be undesirable in some patients. In addition, a larger single device may have a greater likelihood of migration which would be reduced if the same total prosthesis volume was delivered in two bulking devices each having half the cross-sectional area of the single larger device.

The bulking device 16 is preferably flexible and has a high degree of softness which approaches that of the native mucosal tissue, to minimize trauma to the adjacent tissue. The material of the bulking device 16 is thus preferably soft enough so that is incapable of exerting sufficient localized pressure to cause pressure necrosis. A localized pressure in excess of about 70 mm Hg gives rise to a risk of localized tissue necrosis. As will be understood by those of skill in the art, the configuration of the bulking device 16 (e.g. sharp edges, etc.) operates in cooperation with the softness of the construction materials to optimize the compatibility of the bulking device. A smooth, blunt a traumatic outer surface is preferred.

One suitable bulking device construction comprises the use of an inflatable pillow or balloon, partially or completely filled with a liquid or semi-liquid, which allows one end to be compressed by peristaltic compression and the other end to expand bulbously. The ability of the volume of the bulking device to flow from one end of the bulking device to the other and back permits the passage of a peristaltic wave, as will be appreciated by those of skill in the art in view of the disclosure herein. Suitable elastomeric balloons comprise material such as silicone, latex, urethane, and others as will be understood by those of skill in the art.

In addition to being soft, the bulking device in some embodiments may also be compressible. This enables the filler and body of bulking device 16 to expand when nothing is passing through the LES, but compress, for example, to no more than about 4 mm and in another embodiment to no more than about 2 mm in radial thickness during swallowing. After swallowing, the filling material and body will desirably rebound back to facilitate the LES closure function. In this manner, the bulking device 16 can cooperate with any residual function of the LES to minimize the occurrence of reflux.

In embodiments of a bulking device 16 that comprise an outer surface which encloses a filler therein, the outer surface may be homogeneous with the filler, or may be the surface of a dissimilar material in the form of a flexible wall to encapsulate the filler. A homogeneous material outer wall may be provided with a different physical property than the filler, such as by heat treatment, solvent exposure or the like to provide a barrier coating around the filler.

The esophageal bulking device 16 can be manufactured as a unitary or multi-component structure in a variety of ways as will be appreciated by those of skill in the art in view of the disclosure herein. The bulking device 16, for example, may be a unitary structure molded as a single piece of biocompatible foam material such as silicone foam or polyurethane foam, or may be cut from a block of such foam. Such foam parts can be made with an outer skin of porous foam that facilitates tissue ingrowth.

Alternatively, the esophageal bulking device 16 can comprise a body having at least two components connected together and can be made, for example, by positioning an outer sleeve or layer of porous material such as expanded polytetrafluoroethylene (PTFE) or other tissue ingrowth material around the filler by either a simple filling operation or by bonding the two materials together. If expanded PTFE is used, a PTFE surface etching step prior to bonding with a silicone based glue may be performed. Alternatively, a process of gluing by simultaneously compressing and heating a stack-up of foam, glue and PTFE can be employed. The outer layer may be secured to the bulking device 16 in any of a variety of manners, such as by solvent bonding, thermal bonding, adhesives, and others as will be apparent to those of skill in the art in view of the disclosure herein.

Other embodiments of the esophageal bulking device 16 include those that have surface textures, coatings or structures to resist migration. In general, the entire outer surface of the outer layer or filler can be coated or textured to facilitate tissue attachment such as by cellular ingrowth. The resulting attachment surface can be integral with the bulking device 16 or can be directly or indirectly connected to the bulking device 16 so that the device 16 can be positioned and retained in the desired position within the esophageal wall. The outer surface may additionally, or alternatively, be provided with any of a variety of tissue retention structures such as hooks, barbs, tacks, clips, sutures, staples, tissue adhesives, attachment strips, attachment spots, attachment connectors, or other attachment means which will be understood by those of skill in the art in view of the disclosure herein.

Delivery of the esophageal bulking device 16 below the mucosa can be accomplished in any of a variety of ways, as will be apparent to those of skill in the art in view of the disclosure herein. Delivery systems can be specially constructed or assembled from existing endoscopic and other surgical tools to accomplish the basic delivery steps.

In general, the delivery site for a particular patient is identified, such as by endoscopy and manometry. Tissue adjacent to the delivery site is preferably immobilized to permit a puncture or incision to be made. Immobilization of the esophageal lining may be accomplished by grasping the tissue utilizing forceps, such as those which may be advanced through a working channel on an endoscope. Alternatively, a vacuum may be applied to a lumen through an endoscope to immobilize the tissue.

Using counter-traction on the tissue applied by way of the tissue grasper or vacuum, the mucosa is pierced to enable insertion of the prosthesis. The mucosal layer may be pierced in a variety of ways, as will be recognized in the art. In accordance with one aspect of the present method, a needle is utilized to pierce the mucosa and create a blister by injecting a volume of fluid such as saline solution.

Once an aperture has been formed in the mucosa, a pouch is formed in the submucosa. The pouch may be formed by liquid infusion to enlarge the blister discussed above. Alternatively, any of a variety of blunt tools may be utilized to achieve a blunt dissection in the submucosa or between adjacent tissue planes to form a pouch for receiving the prosthesis. Alternatively, an inflation device, such as a balloon, may be specially shaped for insertion and inflation to separate submucosal tissue and provide a submucosal pouch.

Following formation of a submucosal pouch, one or more bulking devices 16 are introduced therein. The bulking device 16 may be inserted by way of a grasper, clamshell deployment device, or other tools. Depending upon the shape and compliancy of the bulking device 16, the bulking device 16 may be deployed from the distal end of a tubular element, such as by advancing an axially moveable core to push the prosthesis from the distal end of the tube. One or more pull elements such as wires, strings or tabs may be provided on the bulking device 16, so that a distally extending pull element may be advanced into the pouch under distal force using a grasper, or a second mucosal puncture inferior to the primary mucosal puncture may be provided through which to pull a pull element, thereby advancing the bulking device 16 inferiorly into the pouch.

Following placement of the bulking device 16 into the submucosal pouch, the mucosal opening closes naturally or can be closed using any of a variety of closure techniques. A conventional suture, ligating bands or staples or other clips, may be utilized endoscopically, as will be understood in the art. Alternatively, a topical glue or other adhesive patch may be utilized to close the opening in the mucosa.

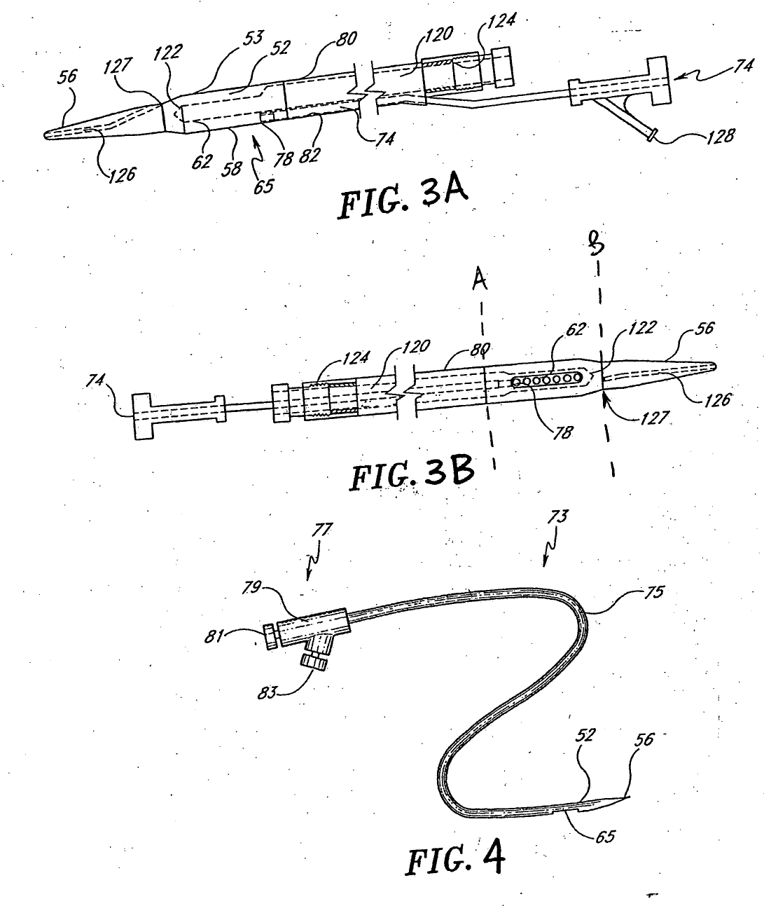

Referring to FIGS. 3A and 3B, there is illustrated a side elevational view of an overtube embodiment adapted to be advanced over the distal end of an endoscope and pulled proximally like a sheath to adapt the endoscope for the present application. In an alternate application, discussed in connection with FIG. 4, infra, the distal end configuration is on an access catheter without an associated endoscope.

In the illustrated embodiment, a flexible tubular sheath 80 extends proximally from the distal end assembly 56. In one embodiment, tubular sheath 80 is dimensioned to receive and closely fit a standard endoscope. For example, one common endoscope that can be used with the present invention has an outside diameter of about 9.8 mm. The corresponding lumen 120 in tubular sheath 80 has an inside diameter of about 11 mm and an axial length within the range of from about 60 cm to about 120 cm. Tubular sheath 80 may be made of any of a variety of polymeric materials which are conventional in the catheter manufacturing arts. The tubular sheath 80 may be integrally formed with or bonded to the side wall 52 of midsection 53 and other structural components of the distal end 56 assembly. In one embodiment, tubular sheath 80 comprises poly vinyl chloride (PVC), having a wall thickness of about 0.050 inches.

A proximally extending deployment lumen 74 is defined within a tubular wall 82. In this construction, the tubular wall 82 extends in parallel with at least a portion of the endoscope, so that the deployment catheter or other deployment device need not be advanced through a working channel in the endoscope. The deployment lumen 74 terminates in a distal end 78 which directs the deployment device or piercing device into tissue which has been drawn into the cavity 65. Proximally, the deployment lumen 74 may terminate in a manifold, which may additionally be provided with a flush port 128 if desired.

The central lumen 120 for receiving an endoscope extends between a distal end 122 in midsection 53 and a proximal endoscope seal 124. In one embodiment, a distal tip 56 preferably extends beyond the midsection 53, to permit atraumatic introduction of the device. Distal tip 56 is preferably provided with a guidewire lumen 126, to facilitate over the wire placement. Guidewire lumen 126 can extend proximally throughout the length of the device, or exit at a proximal side port 127 along the side of the device such as at about the proximal end of the distal tip 56 section.

In one embodiment of the overtube illustrated in FIGS. 3A and 3B, the tubular sheath 80 and distal end 56 assembly have an overall length on the order of about 35 inches. In another embodiment, the overall length is about 34.8 inches. The outside diameter of the overtube is less than about 1 inch. In another embodiment, the diameter of the overtube is about 0.7 inches. The depth of the tissue cavity between the tissue opening 58 and tissue stabilizing surface 62 is about 0.2 inches. Any of these dimensions may be varied, to accommodate different functional characteristics and other clinical objectives of the device, as will be apparent to those of skill in the art in view of the disclosure herein.

Referring to FIG. 4, there is illustrated a side elevational schematic view of another embodiment of the submucosal delivery device in accordance with the present invention. The delivery device 73 is provided with an elongate flexible body 75, having a proximal end 77 and a distal end 56. Proximal end 77 is provided with a manifold 79 which may have any of a variety of features depending upon the functionality of the device. In the illustrated embodiment, the manifold 79 is provided with a deployment device access port 81. Access port 81 is in communication with a deployment lumen (not depicted in FIG. 4). The deployment lumen extends throughout the length of flexible body 75, and communicates with a distal end at or near the tissue cavity 65 as has been discussed.

The proximal manifold 79 is further provided with a vacuum port 83. Vacuum port 83 is in communication with a vacuum lumen (not illustrated) extending throughout the length of flexible body 75, and communicating with the tissue cavity 65 through one or more apertures as has been discussed above.

The embodiment of the delivery device illustrated in FIG. 4 may be utilized without direct visualization. In one embodiment, a sensor can be included to indicate to the clinician when sufficient tissue has been drawn into the cavity 65. Any of a variety of sensors may be devised for this purpose. In one embodiment, a pressure sensor is placed in communication with the vacuum lumen. When sufficient tissue has entered the cavity 65 to occlude the one or more vacuum ports 83, a change in pressure will be perceivable in the vacuum lumen. Similarly, any one or combination of visual, audible, or tactile feedback can be provided to a clinician, indicating that a sufficient volume of tissue has entered the cavity 65. Alternatively, any of a variety of physical pressure gages can be provided in the cavity, and utilized to detect the presence of tissue therein.

The deployment catheter 73 may be used in any of a variety of ways, and dimensions and materials may be modified to accommodate the specific intended use. For example, the deployment device 73 can be utilized to access a treatment site in the esophagus through either the nose or the mouth. Particularly in an application intended to be advanced through the nose, the distal end 56 is provided with an elongate tapered atraumatic tip to enable the device to deflect off the soft palate with minimal trauma. Distal tips having an axial length within the range of from about 1 inch to about 5 inches are contemplated, and may be constructed (e.g., molded) from polyurethane or other soft material.

For either nasal or oral access, the tubular body 75 may have a diameter of within the range of from about 5 mm to about 20 mm, and an axial length within the range of from about 70 cm to about 120 cm. Tubular body 75 may be constructed in any of a variety of manners well known in the catheter construction arts. For example, much of the length of tubular body 75 may comprise a two lumen extrusion with additional lumen (e.g. a guidewire lumen) being provided if additional functionalities are desired. The extrusion may comprise any of a variety of materials such as high density polyethylene (HDPE), polytetrafluoroethylene (PTFE), nylon, acrylic, PVC and other polymers which are well known in the art. The distal end 56 and distal side wall 52 including the tissue cavity 65 may be separately formed and secured to the extrusion in any of a variety of ways, such as through heat melting, adhesive bonding and other techniques which are known to those of skill in the art.

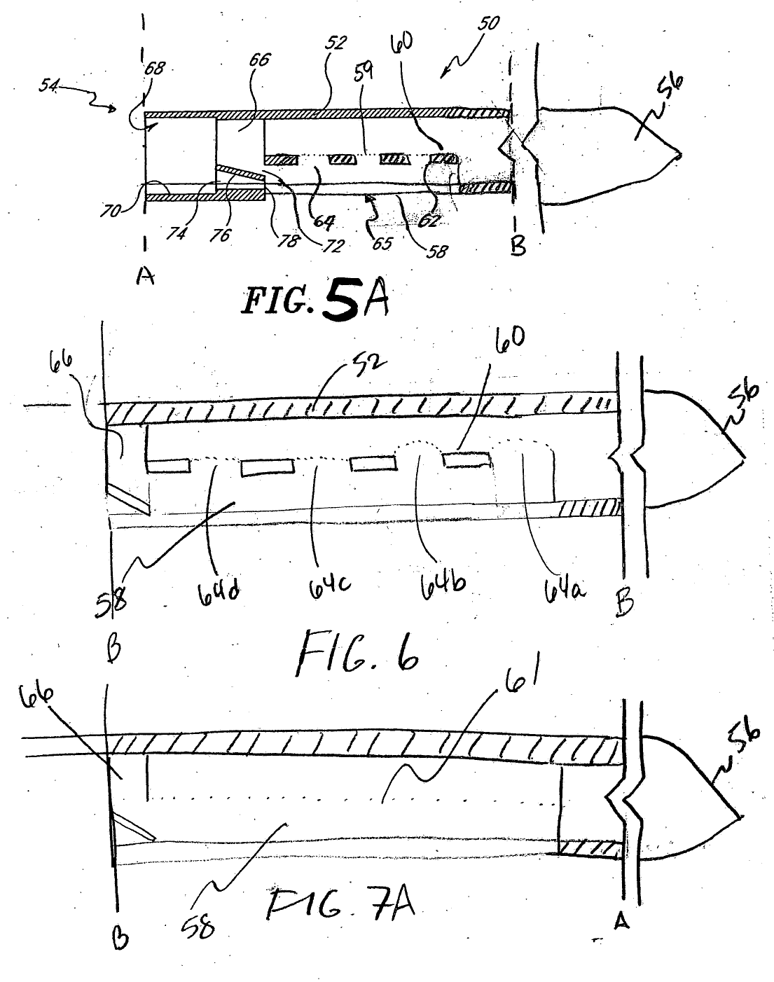

The distal end construction illustrated in FIG. 5A is in the form of a cap which may be integrally manufactured with or subsequently attached to the distal end of an endoscope or other elongate medical device. The cap 50 comprises a sidewall 52 which is generally in the form of tubular element although other constructions may be utilized. Sidewall 52 may be made from any of a variety of biocompatible metals or polymers, such as polycarbonate, acrylic, HDPE, nylon, PTFE, stainless steel, and others well known in the medical device arts. In one embodiment, the cap 50 has an axial length of about 2 inches, an outside diameter of about 0.6 inches, and side wall thickness of about 0.06 inches. In another embodiment, the axial length of the cap 50 is about 2.2. inches.

The sidewall 52 extends between a proximal end 54 and a distal end 56. Distal end 56 preferably blunt, to provide an atraumatic surface. The cap 50 is provided with an opening 58, preferably on the side wall 52, for receiving tissue from the target site. In one embodiment, the opening 58 has an axial length of from about 20 to about 50 mm. In another embodiment, the opening 58 has an axial length of from about 25 mm to about 45 mm. In yet another embodiment, the opening 58 has an axial length of about 35 mm. One embodiment of the device has an opening 58 with a width of from about 2 mm to about 10 mm. Another embodiment has an opening 58 with a width from about 3 mm to about 7 mm. Yet another embodiment has an opening 58 with a width of about 5 mm. In one embodiment, a generally rectangular opening 58 is provided to facilitate introduction of an elongate bulking element into the target site. Other configurations for the opening 58 may be utilized, as is discussed below, depending upon the size and shape of the prosthesis. It should also be understood by one of skill in the art that the size of the opening 58 could be varied even further to allow for the introduction of other sized and shaped bulking devices.

A tissue stabilizing or limit surface 62 defines at least a portion of the wall of the cavity. In the illustrated embodiment, surface 62 is on the lateral face of a surface such as on a shelf or plate 60 positioned medially (towards the central longitudinal axis) from the first opening 58. Preferably, the tissue stabilizing surface 62 is provided with one or more apertures 64, which are in communication through the shelf 60 with a vacuum lumen 66 in the cap 50. Vacuum lumen 66 is adapted for coupling to a lumen in the endoscope or other medical device, so that a vacuum may be drawn on vacuum lumen 66.

The tissue stabilizing surface 62 cooperates with the tissue opening 58 to define a tissue receiving cavity 65 in the medical device. The overall shape and volume of the tissue receiving cavity 65 can be varied widely, depending upon the intended application. For example, tissue opening 58 can have a generally rectangular shape as in the illustrated embodiment. Alternatively, the tissue opening 58 can be in the form of an oval or ellipse, or circle, depending upon the length and width of the desired submucosal low pressure zone or prosthesis. In addition, the tissue stabilizing surface 62 can be concave in the direction of tissue opening 58 or generally planar as illustrated. In one embodiment, the tissue stabilizing surface 62 is in the form of a portion of the side wall of a cylinder, such that it has a substantially linear configuration in the axial direction and a radiused curve in the transverse cross-section. Generally conical or hemispherical configurations for tissue stabilizing surface 62 may also be provided, with an aperture 64 at the apex thereof or a plurality of apertures 64 positioned on the tissue stabilizing surface 62. The tissue stabilizing surface 62 may also be provided with friction enhancing structures, such as a plurality of ridges or grooves to minimize sliding of the submucosal tissue along the surface 62.

In use, tissue which is positioned adjacent the tissue opening 58 will be drawn into the tissue opening 58 and adjacent the tissue stabilizing surface 62 in response to application of vacuum through the apertures 64. The tissue stabilizing surface 62 thus acts as a limit, to reproducibly control the amount of tissue drawn into the opening 58 in response to the vacuum.

The cap 50 may be provided at its proximal end 54 with a second opening 68. Second opening 68 is adapted to be coupled to an endoscope or other medical device. For this purpose, an attachment structure 70 such as an annular recess may be provided on the proximal end 54 to facilitate insertion of the distal end of an endoscope (not shown) therein. The cap 50 may be secured to the endoscope in any of a variety of ways, such as by threaded engagement, snap fit or other interference fit structures, adhesives, or other attachment techniques which will be known in the art.

When coupled to an endoscope, a viewing lens on the endoscope is preferably oriented in the cap 50 such that tissue which has advanced into the cavity 65 can be visualized through the endoscope optics. This may be accomplished by constructing the shelf 60 from a transparent material, and/or providing a viewing aperture 72 between the lens on the endoscope and the tissue opening 58.

A working channel on the endoscope is aligned with a deployment lumen 74. Deployment lumen 74 may be defined at least in part by an internal baffle wall 76. In the illustrated embodiment, the baffle 76 is inclined radially outwardly in the distal direction to taper the inside diameter of the deployment lumen 74 in the distal direction. This may assist in guiding the deployment device (discussed in greater detail below) into the tissue which has been drawn into the cavity 65. The deployment lumen 74 has a distal end 78. Deployment lumen 74 is oriented such that an elongate device which is advanced through deployment lumen 74 and out distal end 78 will extend at least partway across the tissue opening 58. A piercing device such as a needle, cautery tip or other cutting structure which is advanced through deployment lumen 74 will enter and travel beneath tissue which is brought into contact with tissue stabilizing surface 62.

Embodiments of the invention include the use of mesh. The mesh covers portions of the tissue stabilizing surface 62 within the opening 58. One embodiment of the invention covers the one or more apertures 64 within the opening 58 (FIGS. 5A, 5B, and 6) and a second embodiment covers a portion of the tissue stabilizing surface 62 (or shelf 60) itself. The mesh functions to both allow the application of vacuum and limit the depth which tissue is engaged into the opening 58. By limiting the depth which tissue is engaged into the opening, the device may be able to more reproducibly control the amount of tissue drawn into the opening 58 and also therefore the depth at which the bulking device is implanted.

In the embodiment depicted in FIG. 5A, the apertures 64 are covered with a barrier 59. The barrier 59 functions to both allow the application of vacuum through the apertures and limit the depth which tissue can advance through the apertures 64. By limiting the depth which tissue can advance through the apertures 64, the barrier 59 may be able to more reproducibly control the amount of tissue drawn into the opening 58 and also therefore the depth at which the bulking device is implanted.

In one embodiment, the barrier 59 is one continuous piece of mesh, web, network or lattice that spans the back surface of the shelf 60 (i.e. the surface of the shelf 60 that is opposite the tissue limiting surface 62). In such an embodiment, there would be a layer of mesh over the apertures 64 and behind the tissue limiting surface 62 that defines at least a portion of the wall of the cavity. In another embodiment, the barrier 59 spans only the apertures 64 and is not continuous. The barrier 59 can be constructed and/or placed within the device as would be known to those of skill in the art having read this specification. For example, the barrier 59 can be formed within the shelf 60, or formed separately and attached after formation. In an embodiment where the barrier 59 can be secured through heat melting, adhesive bonding, or other techniques which are known to those of skill in the art having read this specification.

The barrier 59 can be made of a size of mesh that allows the application of vacuum through the apertures 64 but does not allow an appreciable amount of tissue to be drawn through the mesh. In one embodiment, the barrier 59 can be formed from a material with a nominal opening of about 0.06 inches or less. In another embodiment the barrier 59 can be formed from a material, a mesh for example, with a sieve designation of 1.70 mm (No. 12—nominal sieve opening of 0.0661 inches) or lower. In another embodiment, the barrier 59 can be formed from a material with a sieve designation of 1.70 mm (No. 12), 1.40 mm (No. 14—nominal sieve opening of 0.0555 inches), 1.18 mm (No. 16—nominal sieve opening of 0.0469 inches) 1.00 mm (No. 18—nominal sieve opening of 0.0394 inches) or a material with a similar construction. In yet another embodiment, the barrier 59 can be formed of a material with a sieve designation of 1.40 mm (No. 14), or a material with similar construction. As used with respect to sieve openings, a material with similar construction means a material that has nominal openings that are between the sieve designation above it and below it. For example, a material with similar construction to a material with a sieve designation of 1.40 mm would include material with nominal sieve openings of 0.0660 inches to 0.0470 inches.

The barrier 59 can be made of any of a variety of materials, as would be known to one of skill in the art having read this specification. Examples of materials that can be used to construct barrier 59 include, but are not limited to titanium, stainless steel, nitinol, plastics, polymers, or similar materials. In one embodiment, the barrier is constructed of stainless steel, titanium, or nitinol. In yet another embodiment, the barrier is constructed of stainless steel or titanium.

In an embodiment of the invention such as that depicted in FIG. 5A, the diameter of the apertures 64 can range from about 0.1 mm to about 5 mm. In another embodiment, the diameter of the apertures 64 can range from about 0.1 mm to about 3 mm. In yet another embodiment, the diameter of the apertures 64 can range from about 0.2 mm to about 0.5 mm. In one embodiment of the invention, the apertures have a diameter of about 3 mm.

In embodiments of the invention where the apertures are not circular, the area of the apertures 64 can range from about 0.007 mm² to about 20 mm². Examples of other possible shapes for apertures 64 include, but are not limited to square, rectangle, and ovals. It should also be recognized by one of skill in the art, that not all apertures 64 in a device need have the same size or configuration. For example, a device could be constructed with larger apertures as you go away from the distal end 56. Alternatively, devices could also be constructed with smaller apertures as you go away form the distal end 56. Devices could also be constructed with, for example, some circular apertures and some oval apertures. All of these embodiments, as well as combinations not discussed are contemplated by the invention.

The embodiments depicted in FIGS. 5A and 5B include four apertures 64. One of skill in the art will understand, having read this specification, that less or more apertures 64 can be utilized in a device of the invention. In one embodiment, there are from 3 to 10 apertures. In another embodiment, there are from 4 to 8 apertures. In yet another embodiment, there are from 6 to 8 apertures. In a further embodiment there are 7 apertures. FIG. 5B depicts a bottom view of the device depicted in FIG. 5A. The apertures 64 and barriers 59 can be seen more distinctly from this view, and as mentioned above, different number of apertures 64 and therefore barriers 59 can be utilized in devices of the invention.

In another embodiment of the invention, exemplified by FIG. 6, the material making up the barriers 59 can have different tightness in different apertures 64. for example, in the embodiment depicted in FIG. 6, the apertures designated as 64a and 64b have looser mesh that those depicted as 64c and 64d. Alternatively, other patterns of apertures with mesh of different tightness can also be utilized. In one embodiment of the invention, an example of which is depicted in FIG. 6, apertures closer to the distal end 56 have looser mesh than the other apertures. In yet another embodiment, apertures farthest from the distal end 56 have looser mesh that the other apertures. In a further embodiment, the tightness of the mesh can be varied in other fashions. Embodiments such as these may allow the tissue to be drawn farther into some apertures than others. This could be useful for visualization of the tissue, equalizing the depth of the tissue across the tissue opening, accommodating for variable angels that the device may approach the tissue from, or some combination thereof.

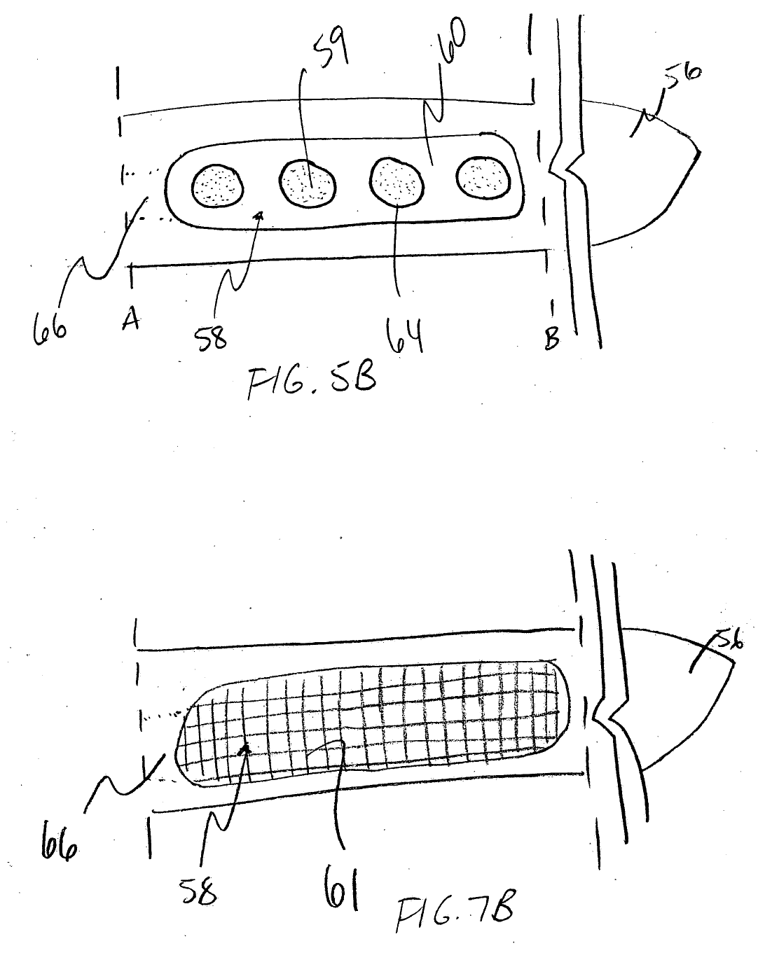

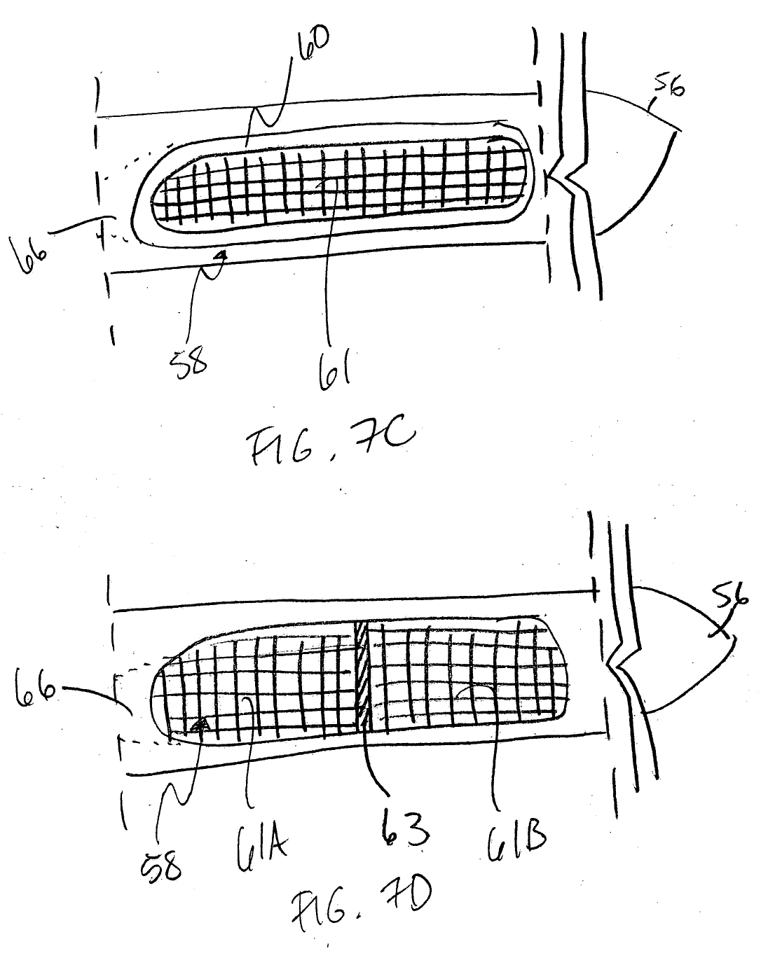

Another embodiment of the invention is depicted in FIG. 7. The embodiment exemplified by FIG. 7 has a different structure than that depicted in FIGS. 5A, 5B, and 6 for the shelf 60. In this embodiment the shelf 60 and barrier 59 are replaced with a screen 61. The screen 61 functions similar to the combination of the shelf 60, the apertures 64, and the barrier 59. The screen 61 can be made of one continuous piece of material that functions to allow the application of vacuum through the screen 61 but does not allow an appreciable amount of tissue to be drawn through the material. The screen 61 can be constructed of materials similar to that which were used for the barrier 59, e.g. mesh.

One embodiment of the invention has a screen 61 which has a width and/or length that are the same size or larger than the opening 58. In embodiments where the screen 61 is larger than the opening 58, the extra material may be used to attach the screen 61 to the area around the opening 58. In another embodiment, the screen 61 has a width and/or length that are smaller than the opening 58. In such an embodiment, the screen 61 is attached to a structure that is similar to the shelf 60 in FIGS. 5A, and 5B. An example of this embodiment is depicted in FIG. 7C with the shelf 60 shown therein.

The size of the opening 58 in embodiments with a screen 61 can be similar to those discussed above with apertures. In embodiments having a screen 61 and a shelf 60, the screen 61 can be smaller, larger, or the same size than the opening 58 itself. In embodiments where the screen 61 is larger or the same size as the opening 58, the screen 61 can overlays or underlay the shelf 60.

Other embodiments of the invention having a screen 61 may also include other structures to provide more tension to the screen 61. An example of such an embodiment can be seen in FIG. 7D. As seen there, one or more cross bars 63 can be included. In such an embodiment, the two portions of the screen 61a and 61b can either be made of the same piece of material (such as mesh) or more than one piece of material.

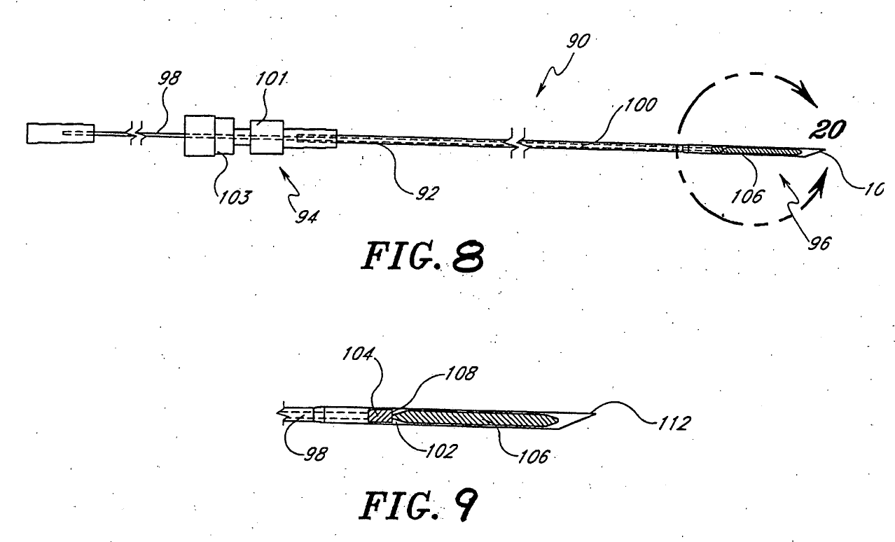

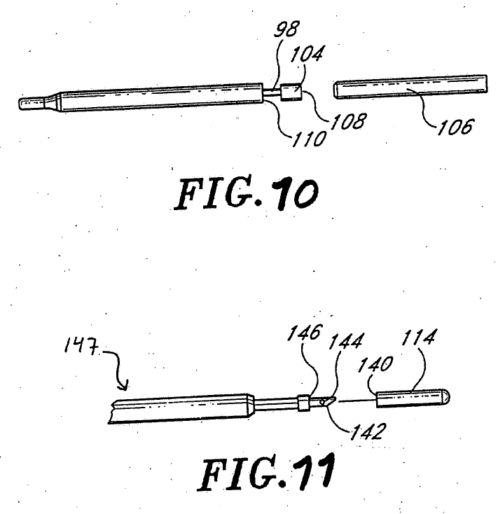

Referring to FIGS. 8 through 11, there are illustrated a variety of deployment devices which may be advanced through the deployment lumen 74 of any of the embodiments previously described. Referring to FIG. 8, a deployment device 90 generally comprises an elongate flexible body 92 having a proximal end 94 and a distal end 96. An inner element 98 is axially movably positioned within an outer tube 100. A proximal locking mechanism 101 is provided on a proximal control 103. Referring to FIG. 9, at the distal end 96, the outer tube 100 comprises a cavity 102 for receiving bulking media 106. A plunger 104 having a distal surface 108 is provided on the distal end of the inner element 98, and is axially movably positionable within the cavity 102. As illustrated in FIG. 10, distal advancement of the inner element 98 causes the plunger 104 to distally deploy the bulking media 106.

The deployment device 90 may be constructed in any of a variety of ways which are known in the catheter construction arts. For example, either or both of the inner element 98 and outer tube 100 may be metal tubes such as hypotubes, dimensioned such that the inner element 98 is axially moveable within the outer tube 100. Alternatively, either or both of the inner element 98 and outer tube 100 may be polymeric extrusions. The inner element 98 may either be a tubular structure or a solid rod.

In an embodiment intended to advance through a deployment lumen 74 having an inside diameter of about 0.100″, the outer tube 100 has an outside diameter of no more than about 0.095″. In one embodiment, the inner element 98 comprises a stainless steel rod, and the outer tube 100 comprises polyimide tube. The deployment device 90 has an axial length within the range of from about 70 cm to about 100 cm, and, in the illustrated embodiment, about 100 cm.

In one embodiment, the length of axial travel of the inner element 98 with respect to the outer tube 100 is sufficient to advance the distal pushing surface 108 to a position beyond the distal end 110 of the cavity 102. In an embodiment intended to deliver a hydrogel bulking device having (an unexpanded) diameter of about 2 mm and axial length of about 20 mm the deployment device 90 is constructed to permit the distal pushing surface 108 on plunger 104 to extend at least about 10 mm beyond the distal end 110 of the cavity 102. The present inventors have determined that permitting the plunger to extend beyond the distal end of the cavity 102 permits the deployment of semi-liquid or gel form bulking agents into a submucosal tissue tract while minimizing the likelihood that trailing threads of the bulking agent will extend proximally through the tissue access pathway upon proximal retraction of the deployment device 90.

In one embodiment, the distal end 96 is provided with a sharpened point 112 for piecing a tissue surface. In this embodiment, an outer protective sheath may be axially moveably carried on the outer tube 100, such that distal advancement of the outer cover 114 will shield the distal pointed tip 112. In an alternative embodiment (FIG. 10), the distal end 96 is blunt.

The deployment or delivery systems disclosed in FIGS. 8 through 11, among other deployment devices, can be used in accordance with the present invention to deliver any of a wide variety of expandable bulking devices or other submucosal prostheses. Suitable bulking devices fall into either of two broad categories. The first are bulking devices which inherently expand to achieve a predetermined configuration. This category includes a wide variety of capsules, foams and hydrogels as have been disclosed previously herein. The second category includes fluids or gels which do not inherently assume any particular shape upon expansion or solidification, but which are able to achieve a predetermined shape in situ when used in accordance with the present invention.

Concerning the first category, a variety of capsules and foams have already been disclosed herein. In addition, a variety of hydrogels may be used such as those disclosed in U.S. Pat. No. 4,943,618 issued Jul. 24, 1990 to Stoy et al., entitled Method for Preparing Polyacrylonitrile Copolymers by Heterogeneous Reaction of Polyacrylonitrile Aquagel, and U.S. Pat. No. 5,252,692 issued Oct. 12, 1993 to Lovy et al entitled Hydrophilic Acrylic Copolymers and Method of Preparation, the disclosures of both of which are incorporated in their entireties herein by reference. Preferably, a hydrogel or other material will be selected which has an expansion ratio on the order of at least about 100% and preferably in excess of 500%, particularly in the transverse direction. Thus, a hydrogel rod having a diameter of about 1 mm when positioned within the cavity 102 will preferably expand to a cross section of about 5 or 6 millimeters or greater in situ. Preferably, the hydrogel or other bulking media will exhibit a sufficient expansion rate that it will expand sufficiently to lock in place before migrating from the deployment site such as through the introduction tissue tract.

Another aspect of the present invention is the ability to introduce a bulking media in a fluid or gel form which possess no inherent ability to achieve a predetermined shape, yet, when used with the devices of the present invention, will produce a bulking structure having a predetermined configuration. This is achieved through the interaction between the devices disclosed herein and the anatomy of the esophagus.

The central esophageal lumen is surrounded by a mucosa layer having a thickness on the order of from about 1.0 to about 2.0 mm. Below the mucosa is the submucosa, having a thickness on the order of about 0.5 to about 1 mm. Below the submucosa is a muscle layer having a variable thickness, generally on the order of about 2 mm to about 3 mm. When mucosa is drawn into the cavity on the delivery device of the present invention, a reproducible low pressure region is created in the submucosa which corresponds in shape to the shape of the mucosa which has been drawn into the cavity. The delivery channel directs a deployment needle or other device through the mucosa and directly into the low pressure region of the submucosa. Thus, in accordance with the present invention, a polymerizable or otherwise hardenable fluid or gel may be injected into the low pressure zone in the submucosa at the target site, and that fluid or gel will assume a shape which is strongly influenced by the shape of the opening 58 and depth of the cavity 65. In addition, the muscle layer is substantially unaffected by the application of vacuum to the mucosa using the devices disclosed herein, because the deployment lumen directs the deployment device generally in parallel to the muscle layer. This minimizes the risk of piercing the muscle layer beneath the submucosa. Among other complications from piercing the muscle layer, the aorta and vagus nerve are positioned closely outside of that layer.

Any of a variety of fluids or gels may be utilized, such as UV curable materials, heat or catalytically initiated polymerizable materials, and others which can be optimized through routine experimentation by those of ordinary skill in the art in view of the disclosure here. Another useful class of bulking media includes biocompatible polymers which are soluble in a biocompatible solvent but insoluble in the submucosal tissue. The biocompatible solvent is soluble or dispersible in the tissue, such that the biocompatible solvent diffuses away from the biocompatible polymer in situ, thereby permitting the biocompatible polymer to precipitate, polymerize, or otherwise harden into a bulking mass. In one embodiment, the bulking media further includes a contrast agent, to permit visualization of the bulking mass during and following implantation of the bulking media. Examples of suitable bulking media are disclosed in U.S. Pat. No. 5,785,642, issued Jul. 28, 1998 to Wallace et al., entitled Methods for Treating Urinary Incontinence in Mammals, the disclosure of which is incorporated in its entirety herein by reference.

Referring to FIG. 11, there is disclosed an alternate distal end 96 for the deployment device 90, adapted to deploy an inflatable balloon 114. The balloon 114 is provided with an opening 140 on a proximal end thereof, which includes a pierceable septum or other sealable closing structure (not illustrated). A tube 142 having at least one inflation aperture 144 thereon is moveably carried within the closure structure during insertion of the deployment device 90. Proximal travel of balloon 114 along the inflation tube 142 is limited by a stop 146. Following placement within a submucosal cavity, the balloon 114 is inflated and inflation tube 142 is proximally withdrawn thereby leaving the inflated balloon in place. Deflation of the balloon is prevented by closure of the pierceable septum. The balloon may be deployed by advancing the delivery system into the submucosal space, retracting covering sheath 147 to expose the balloon 114, inflating the balloon 114 through inflation tube 142 and withdrawing the catheter system leaving the balloon in place.

Although the foregoing invention has been disclosed in terms of certain preferred embodiments, other specific embodiments can be constructed in view of the disclosure herein without departing from the spirit of the scope of the present invention. Accordingly, the scope of the Applicant's invention is to be determined by reference to the attached claims, which are not limited to any of the particular embodiments disclosed herein.

1.) A delivery device for introducing at least one prosthesis beneath a tissue surface comprising:
an elongate body having a proximal end and a distal end;
an opening on the elongate body, the opening being at least partially covered by a mesh; and
a deployment lumen extending through the body, the deployment lumen having a longitudinal axis and a distal end;
wherein the distal end of the deployment lumen is positioned proximal of the opening.

2.) The delivery device according to claim 1, wherein the opening includes a shelf that includes one or more apertures.

3.) The delivery device according to claim 2, wherein the mesh forms a barrier that covers the apertures.

4.) The delivery device according to claim 2 wherein there are from about 3 to about 10 apertures.

5.) The delivery device according to claim 2 wherein there are from about 6 to about 8 apertures.

6.) The delivery device according to claim 2 wherein there are 7 apertures.

7.) The delivery device according to claim 1, wherein the mesh forms a screen in the opening.

8.) The delivery device according to claim 7, wherein the screen has a length and width that are at least the same as the length and width of the opening.

9.) The delivery device according to claim 7, wherein the screen has a length and width that are less than the length and the width of the opening.

10.) The delivery device according to claim 9, wherein the screen rests on a shelf within the opening.

11.) The delivery device according to claim 1, wherein the mesh has a nominal opening of about 0.06 inches or less.

12.) The delivery device according to claim 1, wherein the mesh has a nominal opening of about 0.0555 or less.

13.) The delivery device of claim 1, further comprising a suction lumen extending through the body and communicating with the opening.

14.) A delivery system for delivering an expandable sub-mucosal bulking agent comprising:
a delivery device according to claim 1;
a tubular cannula having a sharpened distal end, positioned within the elongate body, the cannula axially moveable such that the sharpened distal end can penetrate tissue stabilized by the opening of the delivery device; and
a plunger movably positioned within the cannula for ejecting a bulking agent positioned within the cannula,
wherein the plunger is distally advanceable beyond the distal end of the sharpened tip.

15.) A delivery system according to claim 14, wherein the elongate body further comprises a lumen in communication with the opening for applying vacuum to the opening.

16.) A kit comprising:
a delivery system according to claim 14; and
at least one bulking agent.

17.) An overtube for receiving an endoscope, comprising:
an elongate, flexible tubular body, having a proximal end and a distal end and a longitudinal axis;
at least one lumen extending therethrough for receiving an endoscope;
an opening on the body, in communication with the lumen; and
a tissue limiting surface within the body aligned with the opening, formed at least in part by a mesh.

18.) The overtube according to claim 17, wherein the tubular body has a sidewall, and the opening is in the sidewall.

19.) The overtube according to claim 18, wherein the tissue limiting surface comprises a plurality of apertures.

20.) The overtube according to claim 19, wherein there are from about 3 to about 10 apertures.

21.) The overtube according to claim 20, wherein there are from about 6 to about 8 apertures.

22.) The overtube according to claim 17, wherein the mesh forms a screen in the opening.

23.) The overtube according to claim 17, wherein the screen has a length and width that are at least the same as the length and width of the opening.

24.) The overtube according to claim 17, wherein the screen has a length and width that are less than the length and the width of the opening.

25.) The overtube according to claim 24, wherein the screen rests on a shelf within the opening.

26.) The overtube according to claim 17, wherein the mesh has a nominal opening of about 0.06 inches or less.

27.) The overtube according to claim 17, wherein the mesh has a nominal opening of about 0.0555 or less.

28.) A method of stabilizing a tissue site and introducing a prosthesis beneath the tissue, comprising the steps of:
providing an elongate, flexible, tubular device, having a side opening near a distal end thereof;
positioning the side opening at the site;
applying suction to the device to draw tissue laterally into the side opening;
limiting lateral advance of the tissue into the device at least in part by contacting the tissue with mesh; and
introducing a prosthesis beneath the surface at the site while the tissue is in contact with the mesh.

29.) The method according to claim 28, wherein the positioning step comprises introducing the device into the esophagus through the nose.

30.) The method according to claim 28, wherein the positioning step comprises introducing the device into the esophagus through the mouth.

31.) The method according to claim 28, wherein the introducing a prosthesis step comprises introducing a hydrogel.

32.) The method according to claim 31, further comprising the step of permitting the hydrogel to expand at the site.
